(19) United States
(12) Patent Application Publication
(10) Pub. No.: US 2006/0275250 A1
(43) Pub. Date: Dec. 7, 2006

(54) Reactive polymers and copolymers, method of their preparation and their use

(76) Inventors: Vladimir Subr, Meinik (CZ); Karel Ulbrich, Praha (CZ); Blanka Rihova, Praha (CZ)

Correspondence Address: NOTARO AND MICHALOS, 100 DUTCH HILL ROAD, SUITE 110, ORANGEBURG, NY, 10962-2100, US

(21) Appl. No.: 10/564,614

(22) PCT Filed: Jul. 15, 2004
(86) PCT No.: PCT/IB04/52127

§ 371 Date: May 30, 2006

(30) Foreign Application Priority Data
Jul. 16, 2003 (CZ) PV2003-1950

(51) Int. Cl. A61K 38/05; A61K 31/787; A61K 31/785; C08G 63/91

(52) U.S. Cl. 424/78.27; 424/78.3; 525/54.1

(57) ABSTRACT

The solution concerns reactive polymers and copolymers based on N-(2-hydroxypropyl)methacrylamide, which contain reactive thiazolidine-2-thione groups in side chains of the polymers or at the ends of polymer chains. The solution also includes a method of their preparation and their use for synthesis of polymer drugs and conjugates with proteins and preparation of gene delivery systems.

### TECHNICAL FIELD

The invention concerns new reactive polymers and copolymers based on N-(2-hydroxypropyl)methacrylamide, their preparation and use for synthesis of polymer drugs enabling targeted therapy and for modification of biologically active proteins (protein delivery) and preparation of systems for gene therapy.

### BACKGROUND ART

The development of new drugs and drug forms in recent years has been increasingly focused on utilization of polymer substances, in particular water-soluble polymers as drug carriers. An important group of polymer drugs achieving rapid development is the drugs based on N-(2-hydroxypropyl)methacrylamide (HPMA) copolymers. In such polymer drugs the active drug is bonded to the polymer carrier through an enzymatically cleavable oligopeptide sequence, which enables controlled release of the active cytostatic in target (tumorous) cells. The drugs frequently utilize an antibody as a unit specifically targeting the drug on selected organs or cells. The structure, synthesis and properties of such conjugates are described in patents (CZ 278551—J. Kope\check{c}ek, P. Rejmanová, J. Strohalm, R. Duncan, J. B. Lloyd, K. Ulbrich, B. \check{R}íhová, V. Chytrý; U.S. Pat. No. 5,571,785—F. Angelucci, M. Grandi, A. Suarato) [1,2] and in a variety of other publications (K. Ulbrich, V. \check{S}ubr, J. Strohalm, D. Plocová, M. Jelínková, B. \check{R}íhová, Polymeric drugs based on conjugates of synthetic and natural macromolecules I. Synthesis and physico-chemical characterisation: J. Controlled Release 64, 2000, 63-79; B. \check{R}íhová, M. Jelínková, J. Strohalm, V. \check{S}tubr, D. Plocová, O. Hovorka, M. Novák, D. Plundrová, Y. Germano, K. Ulbrich, Polymeric drugs based on conjugates of synthetic and natural macromolecules II. Anticancer activity of antibody or (Fab′)₂-targeted conjugates and combined therapy with immunomodulators, J. Controlled Release 64 (2000) 241-261; J. Kopećek, P. Kopećková, T. Minko, Z. R. Lu, HPMA copolymer-anticancer drug conjugates: design, activity, and mechanism of action: Eur. J. Pharm. Biopharm. 50 (2000) 61-81; K. Ulbrich, J. Strohalm, V. \check{S}ubr, D. Plocová, R. Duncan, B. \check{R}íhová, Polymeric Conjugates of Drugs and Antibodies for Site-Specific Drug Delivery, Macromol. Symp. 103 (1996) 177-192). [3-6].

A survey of results so far achieved is well documented in Kope\check{c}ek et al.: HPMA copolymer-anticancer drug conjugates: design, activity, and mechanism of action: Eur. J. Pharm. Biopharm. 50 (2000) 61-81 [5]. At present some other polymer conjugates are clinically tested. (P. A. Vasey, R. Duncan, S. B. Kaye, J. Cassidy, Clinical phase I trial of PK1 (HPMA co-polymer doxorubicin), Eur. J. Cancer 31 (1995) S193, P. A. Vasey, S. B. Kaye, R. Morrison, C. Twelves, P. Wilson, R. Duncan, A. H. Thomson, L. S. Murray, T. E. Hilditch, T. Murray, S. Burtles, D. Fraier, E. Frigerio, J. Cassidy, Phase I clinical and pharmacokinetic study of PK1 [N-(2-hydroxypropyl)methacrylamide copolymer doxorubicin]: First member of a new class of chemotherapeutic agents—Drug-polymer conjugates, Clin. Cancer Res. 5 (1999) 83-94, P. J. Julyan, L. W. Seymour, D. R. Ferry, S. Daryani, C. M. Boivin, J. Doran, M. David, D. Anderson, C. Christodoulou, A. M. Young, Preliminary clinical study of the distribution of HPMA copolymers bearing doxorubicin and galactosamine, J. Controlled Release 57 (1999) 281-290, A. H. Thomson, P. A. Vasey, L. S. Murray, J. Cassidy, D. Fraier, E. Frigerio, C. Twelves, Population pharmacokinetics in phase I drug development: a phase I study of PK1 in patients with solid tumours: Br. J. Cancer 81 (1999) 99-108. L. W. Seymour, D. R. Ferry, D. Anderson, S. Hesslewood, P. J. Julyan, R. Poyner, J. Doran, A. M. Young, S. Burtles, D. J. Kerr, Hepatic drug targeting: Phase I evaluation of polymer-bound doxorubicin. J. Clin. Oncol. 20, 1668-1676, 2002; N. V. R. Panday, M. J. M. Terwogt, W. W. Huinink et al., Phase I clinical and pharmacokinetic study of PNU 166945, a novel water-soluble prodrug of paclitaxel. Proc. Am. Soc. Clin. Oncol. 17, 742, 1998; M. J. M. Terwogt, W. W. Huinink, J. H. M. Schellens, M. Schot, I. A. M. Mandjes, M. G. Zurlo, M. Rocchetti, H. Rosing, F. J. Koopman, J. H. Beijnen: Phase I clinical and pharmacokinetic study of PNU 166945, a novel water-soluble polymer-conjugated prodrug of paclitaxel. Anti-Cancer Drugs 12, 315-323, 2001; M. Bouma, B. Nuijen, D. R. Stewart, J. R. Rice, B. A. J. Jansen, J. Reedijk, A. Bult, J. H. Beijnen, Stability and compatiblity of the investigational polymer-conjugated platinum anticancer agent AP 5280 in infusion systems and its hemolytic potential. Anti-Cancer Drugs 13, 915-924, 2002; M. M. Tibben, J. M. Rademaker-Lakhai, J. R. Rice, D. R. Steward, J. H. M. Schellens, J. H. Beijnen, Determination of total platinum in plasma and plasma ultrafiltrate, from subjects dosed with the platinum-containing N-(2-hydroxypropyl)methacrylamide copolymer AP5280, by use of graphite-furnace Zeeman atomic-absorption spectrometry, Anal. Bioanal. Chem. 373, 233-236, 2002) [7-15].

At present polymeric cytostatics containing human IgG as targeting unit are in the preclinical testing phase (B. \check{R}íhová, J. Strohalm, K. Kubá\check{c}ková, M. Jelínková, L. Rozprimová, M. \check{S}írová, D. Plocová, T. Mrkvan, M. Ková\check{r}, J. Pokorná, T. Etrych, K. Ulbrich, Drug-HPMA-HuIg conjugates effective against human solid cancer: Adv. Exp. Med. Biol. 519 (2003) 125-143). [16].

The results of clinical testing showed that, e.g., a polymer-based doxorubicin (Dox) is active and less nonspecifically toxic than the free drug. The maximum tolerated dose of PK1 (MTD) is 320 mg/m², which is four-five times more than the clinically used dose of free doxorubicin (60-80 mg/m²), MYD for PK2 is 120 mg/m². In contrast to doxorubicin, no serious changes in cardial functions were observed on application of the polymer drug, although the cumulative dose reached the value 1680 mg/m².

The present synthesis of polymer drugs based on HPMA copolymers, performed according to the procedure described in CZ patent 278551 [1] and many other works is quite complicated and consists of several steps, such as synthesis of HPMA monomers containing reactive ester groups (4-nitrophenyl or succinimidyl esters), synthesis of copolymers containing 4-nitrophenyl (Np) or succinimidyl (Su) esters (polymer precursors), binding of the drug or a targeting unit to polymer carrier and purification and characterization of the polymer drug. The preparation of reactive polymer precursors with 4-nitrophenyl reactive groups is performed by precipitation copolymerization of HPMA with 4-nitrophenyl esters of N-methacryloylated amino acids or oligopeptides in acetone at 50° C. for 24 h. The obtained conversion ranges between 55 and 60%. The polymerization is accompanied by an inhibition period and chain transfer reactions. This hinders controlling the molecular weight in a simple way (initiator or monomer concentration, temperature) and thus also properties of the polymer precursor. The bonding of the drug and targeting unit (antibody) is based on aminolysis of polymeric Np esters with primary amino groups contained in the drug molecule or targeting unit under the formation of the amide bond.

Owing to comparable rates of aminolysis and hydrolysis of polymeric Np esters in aqueous medium, the binding of a cancerostatic such as doxorubicin or another biologically active molecule or targeting unit (galactosamine) (CZ patent 278551) [1] performed in the organic solvent dimethyl sulfoxide (DMSO) and the isolation of the final product is accomplished by precipitation into a large volume of precipitant (acetone-diethyl ether 3:1) and subsequent filtration. The conjugates containing glycoproteins (antibodies) as targeting units are prepared in a two-step process, in which the drug (doxorubicin) is first bonded in an organic solvent (DMSO, DMF) and, after isolation by precipitation of the drug conjugate containing a part of unreacted Np esters, the antibodies are bonded by aminolysis in aqueous solution at constant pH ranging from 8.0 to 8.2, maintained by addition of sodium tetraborate (K. Ulbrich, V. \check{S}tubr, J. Strohalm, D. Plocová, M. Jelínková, B. \check{R}íhová, Polymeric drugs based on conjugates of synthetic and natural macromolecules I. Synthesis and physico-chemical characterisation: J. Controlled Release 64, 2000, 63-79) [3].

In the same way, other biologically active proteins or oligopeptides are modified with soluble polymers based on HPMA copolymers (enzymes such as BS RNase, RNase A, cyclosporin A, lecirelin—K. Ulbrich, J. Strohalm, D. Plocová, D. Oupický, V. \check{S}ubr, J. Sou\check{c}ek, P. Pou\check{c}ková, J. Matou\check{s}ek, Poly[N-(2-hydroxypropyl)methacrylamide] conjugates of bovine seminal ribonuclease. Synthesis, physicochemical and biological properties: J. Bioactive Compat. Polym. 15 (2000) 4-26; J. Sou\check{c}ek, P. Pou\check{c}ková, M. Zadinová, D. Hlou\check{s}ková, D. Plocová, J. Strohalm, Z. Hrkal, T. Oleár, K. Ulbrich, Polymer conjugated bovine seminal ribonuclease inhibits growth of solid tumors and development of metastases in mice: Neoplasma 48 (2001) 127-132; J. Sou\check{c}ek, P. Pou\check{c}ková, J. Strohalm, D. Plocová, D. Hlou\check{s}ková, M. Zadinová, K. Ulbrich, Poly[N-(2-hydroxypropyl)methacrylamide] conjugates of bovine pancreatic ribonuclease (RNase A) inhibit growth of human melanoma in nude mice: J. Drug Targeting 10 (2002) 175-183; B. \check{R}íhová, A. Jegorov, J. Strohalm, V. Mat'ha, P. Rossmann, L. Forn [symbol] sek, K. Ulbrich, Antibody-Targeted Cyclosporin A: J. Controlled Release 19 (1992) 25-39; K. Ulbrich, V. \check{S}ubr, J. Lidický, L. Sedlák, J. Pícha, Polymeric conjugates of lecirelin with protracted activity and their use, CZ Patent 288 568 (2001)) [17-21] or polyelectrolyte DNA (or plasmid) complexes (K. D. Fisher, Y. Stallwood, N. K. Green, K. Ulbrich, V. Mautner, L. W. Seymour, Polymer-coated adenovirus permits efficient retargeting and evades neutralising antibodies: Gene Ther. 8 (2001) 341-348) [22].

All these syntheses are accompanied by hydrolysis of a part of Np or Su ester groups of the polymer and thus to a decreased ability of the polymer to react with the protein or with another biologically active substance and lead to the product whose structure is complicated and difficult to define.

The aim of the present invention is to provide new reactive polymers and copolymers of HPMA containing reactive thiazolidine-2-thione groups, which are simple to prepare, for synthesis of polymer drugs, modification of biologically active proteins and preparation of gene delivery systems.

### DISCLOSURE OF THE INVENTION

The subject of the present invention is reactive N-(2-hydroxypropyl)methacrylamide-based polymers and copolymers for preparation of polymer drugs, modification of biologically active proteins and preparation of gene delivery systems. They are characterized by the presence of reactive thiazolidine-2-thione groups. The groups can be located, according to the invention, on side chains of a polymer or copolymer or at the end of the polymer chain. The preferred embodiment of to the invention is represented by reactive copolymers consisting of 30-3000 monomer units linked in a polymer chain, out of which 60-99.8% are N-(2-hydroxypropyl)methacrylamide units and the rest is reactive monomer units based on N-methacryloylated amino acids or oligopeptides containing reactive thiazolidine-2-thione groups of the general formula Ma-X-TT, where X is an amino acid or oligopeptide, the amino acid being selected from a group including 6-aminohexanoic acid, 4-aminobenzoic acid and β-alanine, and the oligopeptide is selected from a group including GlyGly, GlyPhe, GlyPheGly, GlyLeuGly, GlyLeuLeuGly, GlyPheLeuGly, Gly-DL-PheLeuGly, and GlyLeuPheGly.

A further characteristic of the present invention is the reactive polymer consisting of 20-150 monomer units linked into a polymer chain composed of 100% of N-(2-hydroxypropyl)methacrylamide units and bearing a (3-sulfanylpropanoyl)-thiazolidine-2-thione grouping at the chain end.

The present invention further includes reactive copolymers consisting of 20-150 monomer units linked in a polymer chain composed of 95-99.9% of N-(2-hydroxypropyl)methacrylamide units and 0.1-5% of N-methacryloylated doxorubicin oligopeptides, where the oligopeptides are selected to advantage from the group of GlyPheGly, GlyLeuGly, Gly-DL-PheLeuGly, GlyPheLeuGly, GlyLeuPheGly and GlyLeuLeuGly bearing the (3-sulfanylpropanoyl)-thiazolidine-2-thione grouping at the chain end.

Another preferred embodiment of the invention is reactive polymers consisting of 20-2000 monomer units linked in a polymer chain composed of 100% of N-(2-hydroxypropyl)methacrylamide units and bearing a (4-cyanopentanoyl)-thiazolidine-2-thione grouping at the chain end.

A further characteristic of the invention is reactive copolymers consisting of 20-2000 monomer units linked in a polymer chain composed of 95-99.9% of N-(2-hydroxypropyl)methacrylamide units and 0.1-5% of N-methacryloylated doxorubicin oligopeptides, where the oligopeptides are selected to advantage from the group of GlyPheGly, GlyLeuGly, Gly-DL-PheLeuGly, GlyPheLeuGly, GlyLeuPheGly and GlyLeuLeuGly bearing the (4-cyanopentanoyl)-thiazolidine-2-thione grouping at the chain end.

The present invention further includes reactive monomer units based on N-methacryloylated amino acids or oligopeptides, which contain reactive thiazolidine-2-thione groups of the general formula Ma-X-TT, where X is an amino acid or oligopeptide and the amino acid is selected from the group including 6-aminohexanoic acid, 4-aminobenzoic acid and β-alanine, the oligopeptide is selected to advantage from the group including GlyGly, GlyPhe, GlyPheGly, GlyLeuGly, GlyLeuLeuGly, GlyPheLeuGly, Gly-DL-PheLeuGly and GlyLeuPheGly and TT represents the thiazolidine-2-thione group, suitable for preparation of reactive polymers.

The method od preparation of reactive polymers and copolymers according to the invention consists in subjecting to solution radical polymerization the monomers selected from a group composed of N-(2-hydroxypropyl)methacrylamide and a N-methacryloylated amino acid or oligopeptide containing reactive thiazolidine-2-thione groups.

A further characteristic of the invention is the method of preparation of reactive polymers and copolymers according to the invention, which consists in that the N-(2-hydroxypropyl)methacrylamide monomer is subjected to precipitation radical polymerization in the presence of 3-sulfanylpropanoic acid as a chain carrier or 2,2′-azobis(4-cyanopentanoic acid) as initiator and the obtained polymer is reacted with 4,5-dihydrothiazole-2-thiol.

The reactive copolymers according to the invention can be prepared by a method consisting in solution radical copolymerization of N-(2-hydroxypropyl)methacrylamide with N-methacryloylated oligopeptide of doxorubicin in the presence of 3-sulfanylpropanoic acid as chain carrier or 2,2′-azobis(4-cyanopentanoic acid) as initiator and the obtained polymer is reacted with 4,5-dihydrothiazole-2-thiol.

The present invention involves the use of the reactive polymers and copolymers according to the invention for preparation of polymer conjugates containing a drug such as doxorubicin or daunomycin and the use of the reactive copolymers for the preparation of conjugates containing a ligand for the receptor on the target cell, such as glycoproteins Ig, IgG, hIgG or monoclonal antibody therapeutical purposes.

A further characteristic of the invention is the use of reactive polymers according to the invention for preparation of hydrophilic-polymer-modified polymer complexes (polyplexes) of DNA or plasmids or adenoviruses as gene delivery systems.

The subject of the invention is reactive polymers (polymer precursors) based on copolymers of HPMA with substituted methacryloylated amides, containing reactive thiazolidine-2-thione (TT) groups, their synthesis and use for preparation of polymer drugs and protein conjugates for therapeutical purposes. The exchange of the ONp groups in HPMA copolymers for reactive TT groups brings a significant improvement, simplification and cheapening of the procedure for preparation of polymer drugs based on HPMA copolymers and also conjugates of the polymers with biologically active proteins and oligopeptides. The preparation of polymer precursors containing reactive thiazolidine-2-thione groups (TT polymers) in side chains can be performed to advantage by solution polymerization in dimethyl sulfoxide. Due to a higher polymerization rate, 70-80% conversions can be obtained already after 7-h polymerization (with polymeric Np esters, 50-60% conversions can be achieved not earlier than after 24 h). The required molecular weight of a polymer precursor is not affected by the reactive comonomer content as in the case of Np esters, being controlled by both the monomer and initiator concentrations and polymerization temperature in a wide range of molecular weights.

The preparation of semitelechelic poly(HPMA) precursors containing reactive thiazolidine-2-thione groups (TT polymers) at the ends of polymer chains proceeds in two steps. In the first step semitelechelic poly(HPMA) containing end carboxylic groups are prepared by precipitation radical polymerization in acetone at 50° C. performed for 24 h in the presence of 3-sulfanylpropanoic acid as chain carrier (K. Ulbrich, V. \check{S}ubr, J. Strohalm, D. Plocová, M. Jelínková, B. \check{R}íhová, Polymeric drugs based on conjugates of synthetic and natural macromolecules I. Synthesis and physico-chemical characterisation: J. Controlled Release 64, 2000, 63-79) [3] or by precipitation radical polymerization in acetone at 50° C. for 24 h using 2,2′-azobis(4-cyanopentanoic acid) as initiator (T. Etrych, J. Strohalm, K. Ulbrich, M. Jelínková, B. \check{R}íhová, Targeting of Polymer-drug Conjugates with Antibodies. Effect of the Method of Conjugation: 5th International Symposium On Polymer Therapeutics, Cardiff, Great Britain, 2002, Abstracts, p. 65) [24]. By subsequent reaction of the end carboxylic groups with 4,5-dihydrothiazole-2-thiol in the presence of N,N-dicyclohexylcarbodiimide (DCC) in dimethylformamide (DMF), the reactive polymer precursor is prepared.

Semitelechelic HPMA-Dox polymer precursors, containing reactive thiazolidine-2-thione groups (TT polymers) at the ends of polymer chains and doxorubicin in side chains can be prepared by 24-h solution radical polymerization of HPMA and N-methacryloylated oligopeptides of doxorubicin (GlyPheGly, GlyLeuGly, Gly-DL-PheLeuGly, GlyPheLeuGly, GlyLeuPheGly a GlyLeuLeuGly) in methanol at 50° C. in the presence of 3-sulfanylpropanoic acid as chain carrier [3] or by solution radical polymerization of the above-mentioned comonomers in methanol at 50° C. for 24 h using 2,2′-azobis(4-cyanopentanoic acid) as initiator [24] and subsequent reaction of the end carboxylic groups with 4,5-dihydrothiazole-2-thiol in the presence of N,N′-dicyclohexylcarbodiimide (DCC) in DMF.

Polymer precursors according to the invention, containing reactive TT groups are characterized by a considerable difference between aminolysis and hydrolysis rates in aqueous medium (FIG. 1), which makes it possible to perform binding of drugs and biologically active substances in a single reaction step. Furthermore, the process including the drug binding can be performed in aqueous medium, which leads to a considerable simplification and cheapening of the preparation of polymeric cytostatics and polymer-protein conjugates. Exclusion of the use of large amounts of inflammable solvents (diethyl ether, acetone) in the synthesis is not only environment-friendly but also manifests itself by lower production costs and in simpler securing safety of the production of drug preparations. A comparison of preparation of polymer conjugates is schematically depicted in FIG. 2. FIG. 1 documents the fact that rapid binding of the drug or protein to polymer preferably occurs by aminolysis of the substances and undesirable hydrolysis is strongly suppressed.

### FIGURES

FIG. 1 shows a comparison of rates of hydrolysis and aminolysis of copolymers P-Akap-TT and P-GlyGly-ONp in HEPES buffer at pH 8.0, ♦ P-Akap-TT hydrolysis, ⋄ P-Akap-TT aminolysis, ▴ P-GlyGly-ONp hydrolysis, Δ P-GlyGly-ONp aminolysis.

Individual steps in the synthesis of polymer conjugates containing the drug and glycoprotein from starting monomers HPMA and N-methacryloylated amino acids and oligopeptides containing reactive TT and ONp groups are given in FIG. 2.

FIG. 3 demonstrates the activity of the classic and star BS-RNase conjugates in the treatment of human melanoma in nu-nu mice.

FIG. 4 shows the survival time of experimental mice in the therapeutic mode of administration of the conjugate prepared according to Examples 5 and 6 of the present invention.

General structures of reactive compounds according to the invention are given in FIGS. 5 and 6, where structure I represents a monomer of general formula Ma-X-TT, structure II copolymers with the reactive thiazolidine-2-thione group in side chain, structures III and V the polymers with reactive groups at the chain ends and structures IV and VI copolymers with reactive groups at the chain ends.

FIG. 7 shows the structures of the compounds that can be prepared using the reactive polymers according to the invention, where structure VII represents an example of a nontargeted cancerostatic and structure VIII an example of an antibody-targeted cancerostatic.

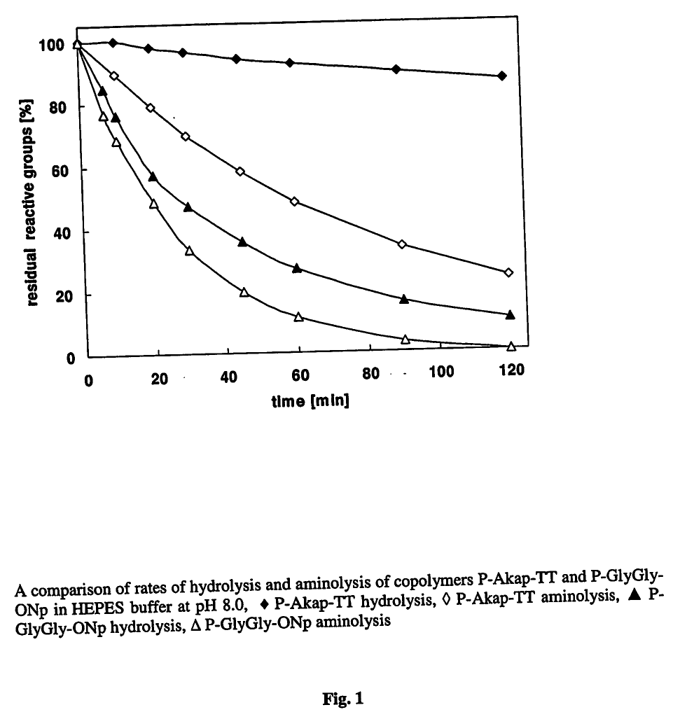

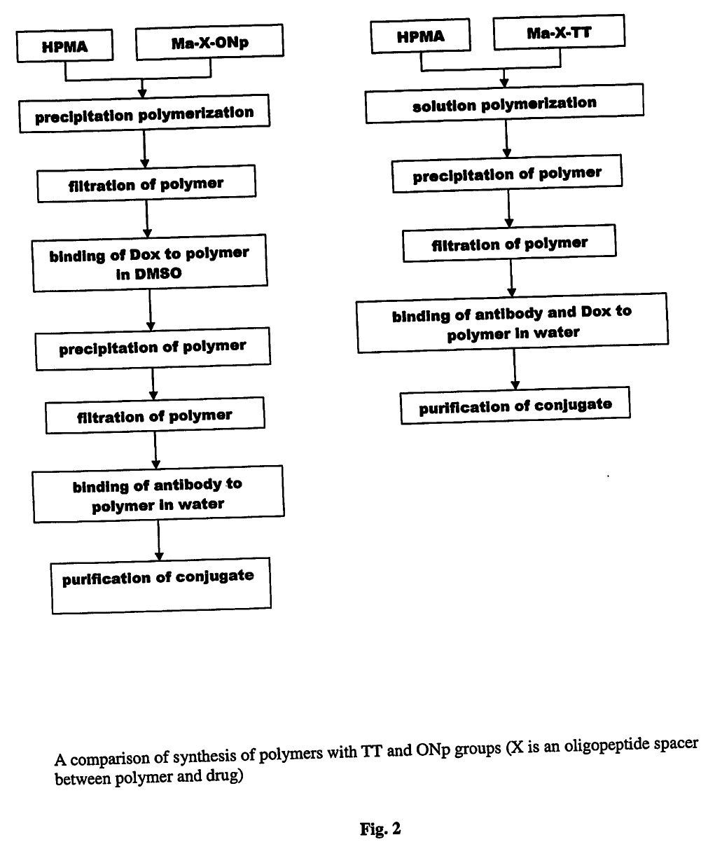

The invention is explained in more detail in the following examples of embodiment, where examples are given of preparation of reactive monomers, of synthesis of reactive polymers (polymer precursors) using reactive monomers and also examples of the use of these precursors for preparation of polymer drugs or conjugates, without being limited to them.

### EXAMPLES

Preparation of Polymer Precursors

The preparation of reactive polymers is performed in two synthetic steps. In the first, monomers are prepared—N-(2-hydroxypropyl)methacrylamide (HPMA) and N-methacryloylated amino acids and oligopeptides containing thiazolidine-2-thione reactive groups (Ma-X-TT, Structure I, FIG. 5). In the second step, the resulting reactive polymers are prepared by radical copolymerization of HPMA with Ma-X-TT (X is an oligopeptide or amino acid).

### Example 1

Reactive TT copolymer with a nondegradable spacer formed by 6-aminohexanoic acid (P-Akap-TT) (Structure II, FIG. 5)

HPMA was prepared by a previously described method [3]. N-Methacryloyl-6-aminohexanoic acid was prepared by methacryloylation of 6-aminohexanoic acid by the Schotten-Baumann reaction [23]. N-methacryloyl-6-aminohexanoic acid (3.0 g, 0.015 mol) and 4,5-dihydrothiazole-2-thiol (1.8 g, 0.015 mol) were dissolved in 35 ml of ethyl acetate. Dicyclohexylcarbodiimide (DCCI) (3.72 g, 0.018 mol) was dissolved in 5 ml of ethyl acetate. Both solutions were cooled to −15° C., mixed and kept at −15° C. for 1 h and further overnight at 5° C. 0.1 ml of acetic acid was added and the reaction mixture was stirred for 1 h at room temperature. The precipitated dicyclohexylurea (DCU) was filtered off. The solution was concentrated in vacuum and again diluted with ethyl acetate. Another portion of the precipitated dicyclohexylurea (DCU) was filtered off. The product was crystallized from a mixture of ethyl acetate-diethyl ether at −15° C., filtered off, washed with diethyl ether and dried in vacuum.

The resulting polymer was prepared by radical copolymerization. 1 g of a mixture of HPMA (95 mol %, 0.90 g) and Ma-Akap-TT (5 mol %, 0.10 g) and 0.133 g of 2,2′-azobis-isobutyronitrile was dissolved in 5.53 g of dimethyl sulfoxide (DMSO) and the solution was charged into a polymerization ampoule. After bubbling the polymerization mixture with nitrogen, the ampoule was sealed and the polymerization was carried out at 60° C. for 6 h. The polymer was isolated by precipitation into 100 ml of an acetone-diethyl ether (1:1) mixture. The polymer was filtered off, washed with acetone and diethyl ether and dried in vacuum. Molecular weight of the polymer, M_{w}=32 400, M_{w}/Mₙ=1.65 and the TT group content was 3.9 mol %. The composition of the copolymer (the content of side chains with TT reactive end groups) can be controlled by the composition of the polymerization mixture in a broad range, molecular weight can be controlled by initiator and monomer concentrations in the charge and polymerization temperature.

### Example 2

The reactive TT copolymer with a spacer formed by a degradable tetrapeptide sequence (P-Gly-DL-PheLeuGly-TT, P-GlyPheLeuGly-TT) (Structure II, FIG. 5)

HPMA and both comonomers, N-methacryloyl-glycylphenylalanylleucylglycines differing in configuration of phenylalanine (L, DL), were prepared by the methods described previously [3]. Both N-methacryloyl-glycylphenylalanylleucylglycine thiazolidine-2-thiones (Ma-GlyPheLeuGly-TT, Ma-Gly-DL-PheLeuGly-TT) were prepared by the reaction of the acid with 4,5-dihydrothiazole-2-thiol in the absence of dicyclohexylcarbodiimide (DCC). Ma-GlyPheLeuGly-OH (2.0 g, 0.00434 mol) and 4,5-dihydrothiazole-2-thiol (0.544 g, 0.00456 mol) were dissolved in 12 ml of N,N-dimethylformamide (DMF). DCC (1.06 g, 0.00514 mol) was dissolved in 5 ml of DMF. The solutions were cooled to −15° C. and mixed. The reaction mixture was kept at −15° C. for 1 h and further at 5° C. for 48 h. The reaction mixture with added 0.1 ml of acetic acid was stirred for 1 h at room temperature. The precipitated DCU was filtered off and the filtrate was concentrated in vacuum. The oily residue was diluted with acetone and the precipitated residual DCU was filtered off. The product, in a mixture of ethyl acetate and acetone (3:1) was purified on a na silica gel column. The fractions corresponding to the product were collected and the solvent was evaporated to dryness in vacuum. The product was then stirred with diethyl ether, filtered off and dried. Copolymerization of HPMA with particular reactive comonomers was carried out under the same conditions as in the case of the copolymer with the Akap spacer. Molecular weight of the polymer M_{w}=33 100, M_{w}/Mₙ=1.63, the TT group content was 8.22 mol %. The copolymer composition (the content of side chains with reactive TT end groups) can be controlled also in this case by the composition of the polymerization mixture in wide range, molecular weight can be controlled by initiator and monomer concentrations in the charge and by polymerization temperature.

Copolymers with TT groups linked to the polymer with glycine, diglycine or β-alanine spacers were prepared analogously. In these cases HPMA and Ma-Gly-OH, Ma-GlyGly-OH and Ma-β-Ala-OH were the starting materials. The synthetic procedures were analogous to the preparation of P-Akap-TT.

### Example 3

Preparation of semitelechelic HPMA polymers containing reactive thiazolidine-2-thione end groups.

A. Semitelechelic poly(HPMA) containing carboxylic end groups were prepared by precipitation radical polymerization in acetone at 50° C. perfomed for 24 h in the presence of 3-sulfanylpropanoic acid as chain transfer agent [3] or by precipitation radical polymerization in acetone at 50° C. for 24 h using 2,2′-azobis(4-cyanopentanoic acid) as initiator [24].

1 g of semitelechelic poly(HPMA) containing carboxylic end groups (Mₙ=5000, 0,0002 mol COOH) was dissolved in 10 ml of DMF and 4,5-dihydrothiazole-2-thiol (0.238 g, 0.002 mol) and DCC (0.413 g, 0.002 mol) was added to the solution. The reaction mixture was stirred for 24 h at room temperature and then reduced in vacuum to a concentration of 15 wt % of the polymer. The reactive polymer was isolated by precipitation in a acetone:diethyl ether mixture (1:1). The polymer was filtered off, washed with acetone, dissolved in methanol and isolated by precipitation in an acetone-diethyl ether (3:1) mixture. The polymer was filtered off, washed with diethyl ether and dried in vacuum (Structures III and V, FIG. 5).

B. Semitelechelic HPMA-Dox copolymers containing carboxylic end groups were prepared by solution radical copolymerization of HPMA and N-methacryloyl-glycylphenylalanylleucylglycyl-doxorubicin in methanol at 50° C. proceeding for 24 h in the presence of 3-sulfanylpropanoic acid as chain transfer agent [3] or by solution radical copolymerization of the above mentioned comonomers in methanol at 50° C. for 24 h using 2,2′-azobis(4-cyanopentanoic acid) as initiator [24].

1 g of semitelechelic polymer HPMA-Dox containing carboxylic end groups (Mₙ=5000, 0.0002 mol COOH) was dissolved in 10 ml DMF and 4,5-dihydrothiazole-2-thiol (0.238 g, 0.0002 mol) and DCC (0.413 g, 0.002 mol) were added to the solution. The reaction mixture was stirred for 24 h at room temperature, then reduced in vacuum to a concentration of 15 wt % of the polymer. The reactive polymer was isolated by precipitation in a acetone:diethyl ether (1:1) mixture. The polymer was filtrered off, washed with acetone, dissolved in methanol and isolated by precipitation in an acetone-diethyl ether (3:1) mixture. The polymer was filtered off, washed with diethyl ether and dried in vacuum. (Structure IV, FIG. 5 and structure VI, FIG. 6).

### Example 4

Preparation of a Nontargeted Polymer Cancerostatic with Doxorubicin in DMSO

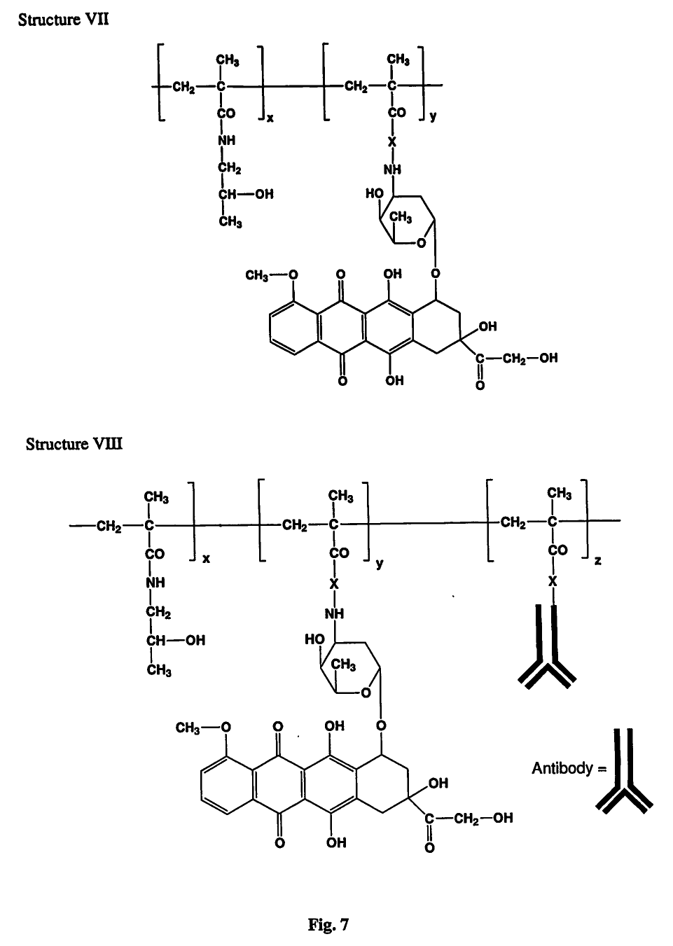

Copolymer P-GlyPheLeuGly-TT (Structure II) (0.15 g) was dissolved in 0.85 ml of DMSO and 0.016 g of Dox.HCl and 0.003 ml of triethylamine were added to the solution. After 1 h stirring, another 0.0012 ml of Et₃N was added and the reaction mixture was stirred for another 1 h. The residual, unreacted TT groups were removed by addition of 0.002 ml of 1-aminopropan-2-ol and the polymer was isolated by precipitation in an acetone-diethyl ether (3:1) mixture. The polymer was filtered off and purified in a methanol solution on a column filled with Sephadex LH-20. The content of bonded Dox was 6.79 wt % (Structure VII, FIG. 7).

### Example 5

Preparation of a Nontargeted Polymer Cancerostatic with Doxorubicin in Water

Copolymer P-GlyPheLeuGly-TT (0.15 g) was dissolved in 1.5 ml of distilled water and 0.016 g Dox.HCl was added to the solution. The reaction mixture was stirred for 2 h at room temperature and pH of the solution was kept at 8.2 (using a pH-stat) by addition of a saturated solution of sodium tetraborate. The remaining, unreacted TT groups were removed by addition of 0.002 ml of 1-aminopropan-2-ol and pH was adjusted to 6.5. The final product in aqueous solution was purified on a column filled with Sephadex G-25 and then lyophilized. The content of bound Dox was 6.51 wt %.

### Example 6

Preparation of a Classic Antibody-Targeted Polymeric Cancerostatic with Doxorubicin (Structure VIII, FIG. 7)

Copolymer P-GlyPheLeuGly-TT (0.1 g, 8.22 mol % TT groups) was dissolved in 5.0 ml of Adriablastina® CS (Pharmacia-Upjohn, a drug form of Dox.HCl, 2 mg Dox/ml of 0.15 M NaCl) and then 35 mg of hIgG (Intraglobin F, Biotest GmbH) in 1.87 ml of distilled water was added. The starting pH 5.0 was adjusted to 8.0 (using a pH-stat) by addition of sodium tetraborate and kept at this value for 1.5 h. Then it was increased to 8.2 and kept for the following 4.5 h. Then 0.002 ml of 1-aminopropan-2-ol was added and pH was adjusted to 6.5. The final product in aqueous solution was purified on a Sephadex G-25 column and lyophilized. The conjugate contained 4.3 wt % of Dox and 29.7 wt % of hIgG. Molecular weight M_{w} of the conjugate was 885 000.

### Example 7

Preparation of an Antibody-Targeted Star-Polymeric Cancerostatic with Doxorubicin

For the preparation of a cytostatic based on star copolymer of HPMA, a semitelechelic copolymer bearing Dox in side chains was used, prepared according to Example 3B. The reaction of the copolymer with antibody was carried out according to the procedure for the synthesis of a star conjugate from a semitelechelic Np ester [3]. The reactions were performed at various copolymer/antibody ratios in the starting mixture and in this way also the product composition (antibody content in the final drug and molecular weight of the product) was controlled. Although both reactions lead to very similar products (Dox content in in the conjugate 4-5 wt %, M_{w}˜500 000), the reaction starting from the TT HPMA copolymer led to higher yields and smaller contents of unreacted (hydrolyzed) polymer in the reaction mixture at the end of the reaction. This makes it possible to set precisely the degree of substitution of the antibody with the polymer by simply changing the weights of both starting reaction components. The purification of the product from the unreacted polymer is then simpler as well.

### Example 8

Preparation of a Classic Conjugate of HPMA Copolymer with Beef Pancreatic RNase (RNase A)

The classic conjugate was prepared by the reaction of the polymer prepared according to Example 2 (P-Gly-DL-PheLeuGly-TT) with RNase A under the same conditions as given in [3]. The RNase A content in the polymer conjugate was determined by amino acid analysis (LDC-Analytical, column with reverse phase Nucleosil 120-3 C₁₈ Macherey Nagel, OPA derivatization [3], purity checked by SDS-PAGE electrophoresis (gradient gel 10-15 Phastsystem (Pharmacia LKB) and the conjugate was characterized by GPC (Superose 6; 0.05 M Tris buffer, pH 8.0).

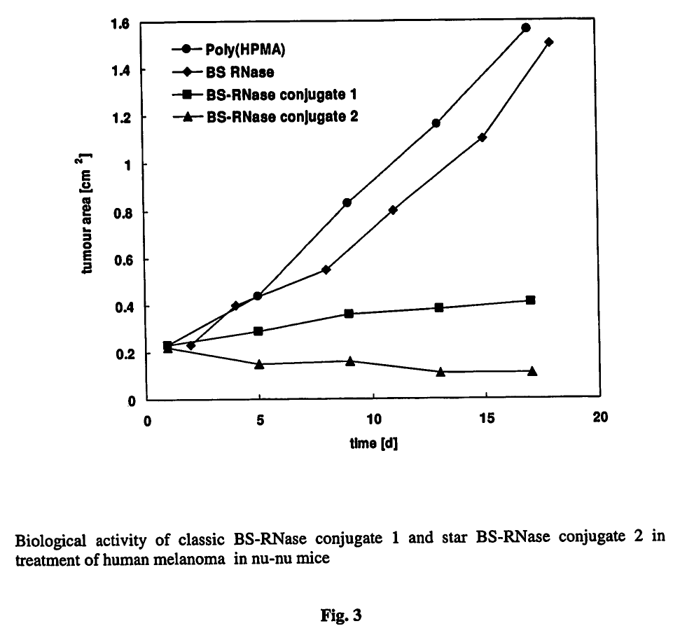

The properties of the conjugate were compared with those of the conjugate prepared from the classic ONp reactive polymer. It was found that physicochemical properties of both conjugates (protein contents, molecular weights) and also biological properties in the treatment of human melanoma in nu-nu mice (FIG. 3) are similar. The synthesis using the reactive polymer according to the invention proceeded faster, a polymer with a smaller content of reactive groups (2 mol %) could be used for obtaining the same product, and in the resulting conjugate no unmodified protein or unreacted polymer was present (the conversion of the reaction of reactive groups was higher).

### Example 9

Preparation of a Star-Like Poly(HMPA) RNase A Conjugate

A star-like poly(HMPA)—RNase A conjugate was prepared from a semitelechelic polymer prepared according to Example 3 by the same procedure as in the synthesis starting from poly(HPMA) with succinimidyl end group [3]. The star conjugate was purified from low-molecular-weight materials by preparative gel chromatography (Sephacryl S300, column 26×600 mm, flow-rate 12.5 ml/h, distilled water). After concentration using an ultrafiltration membrane (PM 30), the product was lyophilized. Comparing the conjugate syntheses using polymers with OSu and TT reactive groups, the latter led to higher reaction yields and much smaller amounts of unreacted (hydrolyzed) polymer in the reaction mixture. The resulting conjugate was active under in vivo conditions equally well as the conjugate prepared from reactive Su ester (FIG. 3).

### Example 10

In Vitro Activity (Cytotoxicity) of Polymeric Doxorubicin Cancerostatics

In vitro cytotoxicity tests were performed by a standard method [4] on ConA-stimulated and nonstimulated mouse T-splenocytes and on a tumour line of mouse T-cell lymphom EL-4. Cytotoxicity was followed by a change of incorporation of [³H]thymidine into cells incubated in a medium containing the tested sample in various concentrations. The cytotoxicity was expressed by the IC₅₀ factor (the substance concentration at which a 50% decrease in proliferation of tested cells is observed). The test results are shown in Table 1. They showed that the properties of the conjugates prepared by the simpler and less expensive method according to the invention are in accord with those prepared by the more demanding classic method.

Table 1

A comparison of cytotoxicity of polymeric Dox cancerostatics prepared from thiazolidine-2-thione (TT) and classic 4-nitrophenyl (ONp) polymers

| | Splenocytes | |
|---|---|---|
| | (ConA) | EL-4 |
| Conjugate | IC₅₀ [μg/ml] | IC₅₀ [μg/ml] |
| Dox | 0.07 | 0.03 |
| P-Gly-DL-PheLeuGly-Dox (TT) | ≧8.00 | ≧8.00 |
| P-Gly-DL-PheLeuGly-Dox (ONp) | 21.5 | 19.1 |
| P-Gly-DL-PheLeuGly-Dox(hIgG) (TT) | ≧8.00 | ≧8.00 |
| P-Gly-DL-PheLeuGly-Dox(hIgG) (ONp) | ˜8.00 | 11.8 |
| P-GlyPheLeuGly-Dox(hIgG) (TT) | ≧8.00 | ≧8.00 |

### Example 11

Comparison of In Vivo Activity of Polymeric Dox Cytostatics Prepared from TT and ONp Polymers

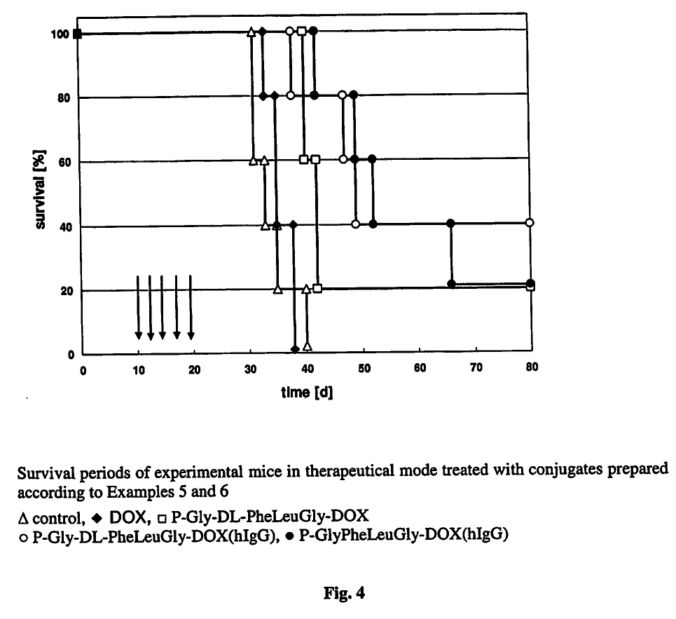

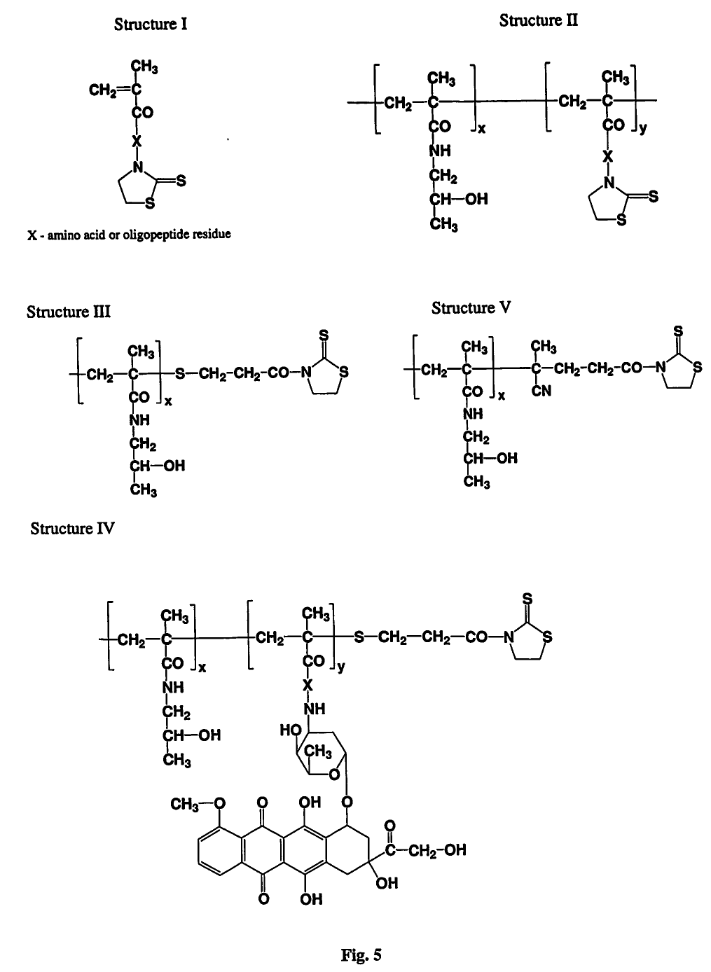

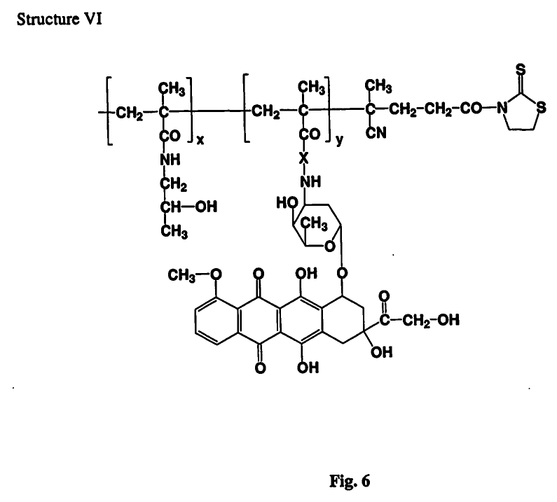

In vivo tests were performed on C57BL/10 strain mice with inoculated cells of mouse T-cell lymphoma EL4. The tumour cells (10⁵) were administered subcutaneously (s.c.) into the right lower half of the dorsal side of mice on day 0. The drug (polymeric cytostatic with a GlyPheLeuGly sequence) was administered in the therapeutic regime (5 mg/kg doses on days 10, 12, 14, 16 a 18 after inoculation). The tumour growth and survival of tested animals were followed. Examples of results are given in FIG. 4. It was proved that in in vivo conditions the activities of both polymeric cytostatics, the classic one prepared from the ONp polymer and the drug prepared by the new method via TT polymers, are identical. The treatment with polymeric cytostatics was considerably more efficient than the classic treatment with commercial doxorubicin.

### Example 12

### Surface Modification of a Polyelectrolyte Complex (Polyplex) of DNA Plasmid with a Hydrophilic Polymer

The polyelectrolyte complex of a polycation of polylysine with DNA (or of a specific plasmid), pLL/DNA, prepared according to [25] was surface-modified with the reactive polymer of structure II and also of structure III. Polymer complex pLL/DNA prepared at the +/− charge ratio 2:1 (molecular weight of the used pLL was 20 000) in HEPES (pH 7.5) at a concentration of 20 μg/ml DNA (5 ml) was mixed with 200 μg of the polymer of structure II or III and the reaction mixture was stirred for 15 min at room temperature. Similarly to ref. [25], 300 μg of PEG-NH₂ modified with a biologically active oligopeptide (SIKVAVS) was added to the reaction mixture and in both cases it was left reacting overnight at room temperature. The unreacted polymer and a possible oligopeptide derivative were removed from the mixture on a concentrator Vivaspin 20 (cut-off 100 000 Da) and the surface-modified, both nontargeted and oligopeptide-targeted complex were used for tests of stability and biological activity. It was shown that the polymer-modified polymer is considerably more stable both in salt solutions and in the presence of blood proteins (albumin). The ability of DNA transfection in vitro was retained.

1. Reactive polymers and copolymers based on N-(2-hydroxypropyl)methacrylamide for preparation of polymeric drugs, modification of biologically active proteins and preparation of gene delivery systems characterized in that they contain reactive thiazolidine-2-thione groups.

2. Reactive polymers and copolymers according to claim 1 characterized in that they contain reactive thiazolidine-2-thione groups in side chains of the polymers or copolymers.

3. Reactive polymers and copolymers according to claim 1 characterized in that they contain reactive thiazolidine-2-thione groups at the ends of polymer chains.

4. Reactive copolymers according to claim 2, characterized in that they consist of 30-3000 monomer units linked in a polymer chain, out of which 60-99.8% are N-(2-hydroxypropyl)methacrylamide units and 0.2-40% are reactive monomer units based on N-methacryloylated amino acids or oligopeptides containing reactive thiazolidine-2-thione groups of the general formula Ma-X-TT, where X is an amino acid or oligopeptide and the amino acid is seloected from a group including 6-aminohexanoic acid, 4-aminobenzoic acid and O-alanine and the oligopeptide is selected from a group including GlyGly, GlyPhe, GlyPheGly, GlyLeuGly, GlyPheLeuGly, Gly-DL-PheLeuGly, GlyLeuPheGly.

5. Reactive polymers according to claim 3, characterized in that they consist of 20-150 monomer units linked in a polymer chain composed of 100% N-(2-hydroxypropyl)methacrylamide units and bearing (3-sulfanylpropanoyl)-thiazolidine-2-thione grouping at the chain end.

6. Reactive polymers according to claim 5, characterized in that they consist of 20-150 monomer units linked in a polymer chain composed of 95-99.9% N-(2-hydroxypropyl)methacrylamide units and 0.1-5% N-methacryloylated oligopeptides of doxorubicinu, where oligopeptides are selected from a group including GlyPheGly GlyLeuGly, Gly-DL-PheLeuGly, GlyPheLeuGly, GlyLeuPheGly and GlyLeuLeuGly, and bearing (3-sulfanylpropanoyl)-thiazolidine-2-thione grouping at the chain end.

7. Reactive polymers according to claim 3, characterized in that they consist of 20-2000 monomer units linked in a polymer chain composed of 100% N-(2-hydroxypropyl)methacrylamide units and bearing (4-cyanopentanoyl)-thiazolidine-2-thione group at the chain end.

8. Reactive polymers according to claim 7, characterized in that they consist of 20-2000 monomer units linked in a polymer chain composed of 95-99.9% N-(2-hydroxypropyl)methacrylamide units and 0.1-5% N-methacryloylated oligopeptides of doxorubicinu, where oligopeptides are selected from a group including GlyPheGly GlyLeuGly, Gly-DL-PheLeuGly, GlyPheLeuGly, GlyLeuPheGly and GlyLeuLeuGly, and bearing (4-cyanopentanoyl)thiazolidine-2-thione group at the chain end.

9. Reactive monomer units based on N-methacryloylated amino acids or oligopeptides for preparation of polymers according to claim 4, characterized in that they consist of N methacryloylated amino acids or oligopeptides containing reactive thiazolidine-2-thione groups of the general formula Ma-X-TT, where X is an amino acid or oligopeptide and the amino acid is selected from a group including 6-aminohexanoic acid, 4-aminobenzoic acid and 0-alanine and the oligopeptide is selected from a group including GlyGly, GlyPhe, GlyPheGly, GlyLeuGly, GlyPheLeuGly, Gly-DL-PheLeuGly, GlyLeuPheGly and TT is a reactive thiazolidine-2-thione group.

10. Method of preparation of reactive polymers and copolymers according to claim 1 characterized in that the monomers selected from the group consisting of N-(2-hydroxypropyl)methacrylamide and N-methacryloylated amino acid or oligopeptide containing reactive thiazolidine-2-thione groups are subjected to radical copolymerization in solution.

11. Method of preparation of reactive polymers and copolymers according to claim 1 characterized in that the monomer N(2-hydroxypropyl)methacrylamide is subjected to precipitation radical polymerization in the presence of 3-sulfanylpropanoic acid as chain carrier or 2,2′-azobis (4-cyanopentanoic acid) as initiator and the obtained polymer is reacted with 4,5-dihydrothiazole-2-thiol.

12. Method of preparation of reactive polymers and copolymers according to claim 6 characterized in that the monomer N(2-hydroxypropyl)methacrylamide is subjected to solution radical copolymerization with a N-methacryloylated oligopeptide of doxorubicine in the presence of 3-sulfanylpropanoic acid as chain carrier or 2,2′-azobis (4-cyanopentanoic acid) as initiator and the obtained polymer is reacted with 4,5-dihydrothiazole-2-thiol.

13. The use of reactive polymers according to claim 1 for preparation of polymer conjugates containing a drug such as doxorubicin and daunomycin.

14. The use of reactive copolymers according to claim 1 for preparation of polymer conjugates containing a protein such as IgG, hIgG and monoclonal antibody.

15. The use of reactive polymers according to claim 1 for preparation of hydrophilic-polymer-modified (“coated”) polymer complexes (polyplexes) of DNA plasmids or adenoviruses as gene delivery systems.

16. Method of preparation of reactive polymers and copolymers according to claim 8 characterized in that the monomer N(2-hydroxypropyl)methacrylamide is subjected to solution radical copolymerization with a N-methacryloylated oligopeptide of doxorubicine in the presence of 3-sulfanylpropanoic acid as chain carrier or 2,2′-azobis (4-cyanopentanoic acid) as initiator and the obtained polymer is reacted with 4,5-dihydrothiazole-2-thiol.
